(19) United States
(12) Patent Application Publication
(10) Pub. No.: US 2026/0045349 A1
(43) Pub. Date: Feb. 12, 2026

(54) HEALTH ANALYSIS ARCHITECTURE

(71) Applicant: Teri Cochrane IP LLC, Reston, VA (US)

(72) Inventor: Teri Cochrane, Reston, VA (US)

(73) Assignee: Teri Cochrane IP LLC, Reston, VA (US)

(21) Appl. No.: 19/292,558

(22) Filed: Aug. 6, 2025

Related U.S. Application Data
(60) Provisional application No. 63/679,841, filed on Aug. 6, 2024.

(51) Int. Cl. G16H 20/30; G16B 25/10; G16H 10/60

(52) U.S. Cl. CPC G16H 20/30; G16B 25/10; G16H 10/60

(57) ABSTRACT

A method may iteratively intaking data corresponding to a patient. A method may apply an epigenetic expression analysis to the data. A method may determine a hierarchy of needs based on the data. A method may apply an iterative kinesiology analysis. A method may generate a bio-individual plan corresponding to the patient based on at least one of the epigenetic expression analysis, the hierarchy of needs, or the iterative kinesiology analysis.

### CROSS REFERENCE TO RELATED APPLICATION

This application claims priority to U.S. Patent Application Ser. No. 63/679,841, filed Aug. 6, 2024, the entire contents of which are hereby incorporated by reference as though fully set forth herein

### BACKGROUND

Understanding disease states and wellness is confined to the lens of direct symptoms and the limited connection known to allopathic and functional medicine. Current protocols are myopic as they operate within partitioned regions of concern and connection relating to human health and wellness.

### BRIEF DESCRIPTION OF THE DRAWINGS

The present disclosure is best understood from the following detailed description when read with the accompanying Figures. It is emphasized that, in accordance with the standard practice in the industry, various features are not drawn to scale. In fact, the dimensions of the various features may be arbitrarily increased or reduced for clarity of discussion.

FIG. 1 is a block diagram of a system of systems, according to one or more examples of the disclosure.

FIG. 2 is a block diagram of the epigenetic expression system of the system of systems of FIG. 1, according to one or more examples of the disclosure.

FIG. 3 is a block diagram of the hierarchy of needs system of the system of systems of FIG. 1, according to one or more examples of the disclosure.

FIG. 4 is a block diagram of the iterative kinesiology system of the system of systems of FIG. 1, according to one or more examples of the disclosure.

FIG. 5 is a block diagram of a bio-individual plan generator, according to one or more examples of the disclosure.

FIG. 6 is a flowchart depicting a method for implementing the system of systems, according to one or more examples of the disclosure.

### DETAILED DESCRIPTION

Illustrative examples of the subject matter claimed below will now be disclosed. In the interest of clarity, not all features of an actual implementation are described in this specification. It will be appreciated that in the development of any such actual implementation, numerous implementation-specific decisions may be made to achieve the developers'specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

Further, as used herein, the article “a” is intended to have its ordinary meaning in the patent arts, namely “one or more.” Herein, the term “about” when applied to a value generally means within the tolerance range of the equipment used to produce the value, or in some examples, means plus or minus 10%, or plus or minus 5%, or plus or minus 1%, unless otherwise expressly specified. Further, herein the term “substantially” as used herein means a majority, or almost all, or all, or an amount with a range of about 51% to about 100%, for example. Moreover, examples herein are intended to be illustrative only and are presented for discussion purposes and not by way of limitation.

Current protocols, while based on scientific, peer reviewed studies, may lack the methodology to produce whole-life, regenerative, sustainable change, because they do not adequately address the multi-dimensional complexities of bio-individuality. Serum labs and functional medicine tests are incomplete without a system that can cut through the noise and identify the body's hierarchy of needs based on an individual's current state of health and genetic tendencies.

Examples provided herein describe a genetically oriented symptom-based approach for untangling a personal algorithm to rebalance the body back to a state of health, vitality and longevity. Protocol recommendations formulated through the rigorous identification of individual genetic tendencies (e.g., single nucleotide polymorphisms, (SNPs)), and the epigenetic expression of the same, environmental conditions, pathogenic loads, and “system of systems” interactions truly address the “why” and offer legitimate solutions. An efficient and effective method to rebalance, heal, and fortify the individual is presented here.

For example, embodiments described herein stand up to novel disease threats as exemplified by the Covid-19 pandemic. The system of systems focuses on the dietary factors that most commonly disrupt metabolism the malabsorption of the “Big Four” (protein, fat/iron, sulfur/histamine/phase I liver detoxification, and oxalates) and observed that the spike protein magnifies these disruptions leading to impaired metabolism. Many suffering from long-haul covid may benefit, not from cutting edge drugs or technologies, but by improving metabolism of the Big 4 with respect to the individual's genetic tendencies and current state of health.

The body will heal itself given the correct conditions. Empowering individuals to understand the intelligence of their body and to restore their health is the ultimate goal of the system of systems. Healthy individuals are catalyzers and create a ripple effect for others in need by raising the level of health, awareness of this system, and consciousness on the planet.

The system of systems encompasses bio-individuality by intaking information, applying a correlation between genetic expression, pathogenic re-expression, environmental and food-based toxins, foods and agents that downregulate metabolic pathways such as liver detoxification, as well as the emotional and physical burdens affecting the body.

Furthermore, the role of food and dietary disruptors is factored into the determination. Amyloids (protein), glyphosates (sulfur/histamine/phase I liver detox), mycotoxins (oxalate and fat), fat, protein, sulfur and oxalate malabsorption can exert considerable influence over epigenetic tendencies and pathogenic reactivation. The system of systems allows for iteration and connective logic to promote lasting health and longevity, as symptomatology, environmental factors, and disease states shift in an individual's health and wellbeing.

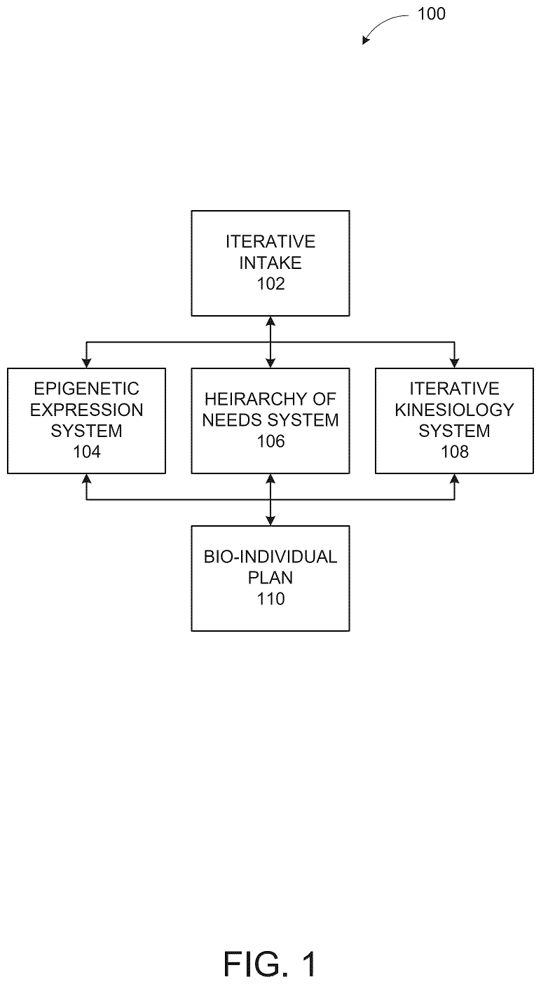

FIG. 1 is a block diagram of a system of systems 100, according to one or more examples of the disclosure. In some embodiments, the system of systems 100 includes an iterative intake 102 to feed data to three interrelated processing components: an epigenetic expression system 104, a hierarchy of needs system 106, and an iterative kinesiology system 108. The output of the system of systems 100 is a bio-individual plan 110.

The epigenetic expression system 104 may intake data corresponding to an individual and provide insight into how and why the body expresses symptomatology and what has contributed to its emergence and resonance. The hierarchy of needs system 106 may be applied based on the iterative intake 102 and/or the output of the epigenetic expression system 104 to provide a component of the bio-individual plan 110 focused on a rebalance of the individual using a deliberate, nuanced, and granular approach to, for example, food and supplementation solutions.

The iterative intake 102 may provide information as input to an application of the iterative kinesiology system 108. In some embodiments, the iterative kinesiology system 108 allows for identification of a source of a health imbalance by measuring and correlating a physical or other response to various stimuli. The iterative kinesiology system 108 may facilitate the creation of a road map of symptomatic expression and signaling impairment as a component of the bio-individual plan 110. Some examples of the bio-individual plan 110 may include reports or other documents or presentations relating to the health and wellness of an individual.

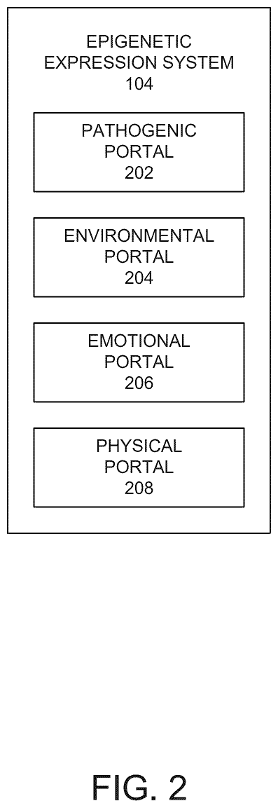

FIG. 2 is a block diagram of the epigenetic expression system 104 of the system of systems 100 of FIG. 1, according to one or more examples of the disclosure. Epigenetics is the science of how behaviors and environment impact genetic expression. Embodiments of the epigenetic expression system 104 relates to phenotypes, or inheritable shifts in gene expression apart from those that the structure of the core genotype, or DNA. Shifts in genetic expression may impact how cells receive and interpret information from genes, and can result in either improved health, or deleterious disease states.

Understanding how genes are tripped may be useful in forming the bio-individual plan 110. For example, this correlative relationship may explain why different bio-individual plans 110 may be generated for individuals with the same condition or symptoms. Where conventional approaches may treat a condition, the system of systems 100 may determine what has affected the body's response in the first place, focusing on the individual. The system of systems 100 applies a pervasive and efficient correlative linking to form the bio-individual plan 110 which can be iteratively applied over time with additional decision points building up to the next testing point, to generate the bio-individual plan 110 which may be customized, actionable, and efficacious to the corresponding individual.

The epigenetic expression system 104 may identify and correlate genetically expressed imbalances and impairments, identifying their root cause, and isolating effective solutions for rebalancing an individual at the epigenetic blueprint level.

The epigenetic expression system 104 may include: a pathogenic portal 202, an environmental portal 204, an emotional portal 206, and a physical portal 208. In some embodiments, the pathogenic portal 202 corresponds to data relating to, for example, bacteria, fungi, parasites, viral, and/or mycotoxic organisms. The environmental portal 204 may correspond to data relating to, for example, food, chemicals, poisons, household toxins, heavy metals, preservatives, supplements, pharmaceuticals, herbicides, fungicides, pesticides, food dyes, body products, and/or household products and the like.

With respect to food, it is important to determine and account for the influence of food intake with respect to the epigenetic expression system 104. Food can trigger an expression or suppression of genes that may have a positive or negative outcome on health. A determination of proper food component via the epigenetic expression system 104 may impact the bio-individual plan 110. The epigenetic expression system 104 may identify, even with improved quality in food choice, unique genetic predispositions and bio-individuality which may require consideration of what foods are doing at the cellular and biochemical level.

The system of systems 100 provides an overview of, for example, protein, fat, sulfur, and oxalates. It is important to note that the ability for these food components to influence disruption in the body is also correlated to the pathogenic portal 202, the environmental portal 204, the emotional portal 206, and the physical portal 208 of the epigenetic expression system 104.

For example, food sources such as domesticated animal proteins, certain vegetables, legumes, nuts or seeds, dairy, and fermented foods may contribute to the progression of disease in certain individuals. Many individuals are being encouraged to eat the “right wrong foods” by educated health care practitioners, doctors, nutritionists, dieticians, health coaches, personal trainers, chiropractors, and acupuncturists. This has lead to confusing and deleterious health effects in the individuals these experts are trying to serve.

Consider, for example, protein. Protein sources are required for the structure, function, and regulation of the body's cells, tissues, and organs. Commercially raised animal proteins repeatedly touted as “healthy” choices include chicken, beef, turkey, or pork. These foods, when raised in inflammatory conditions, may contain misfolded, indigestible protein structures called amyloids. Inflammatory conditions of commercially raised animals may include pen crowding, excessive use of antibiotics, hormones, and chemically treated grain-fed diets. Amyloids are indigestible and contribute to numerous degenerative diseases while promoting biofilm formation and protecting potentially pathogenic bacterial and fungal microorganisms in the gut. Dietary changes, food additives, excessive use of antibiotics, and use of nonsteroidal anti-inflammatory drugs are all factors that may contribute to gut dysbiosis that may lead to pathogenic release of amyloids and LPS. It is important to note that endogenous amyloids are those already existing within a body system that serve as homeostatic mechanisms, while exogenous amyloids are those that come from external sources (i.e. ingestion of commercially raised meats containing amyloids).

The system of systems 100 accounts for the theory that viruses use amyloids to fortify their protective protein coat, while fungi and bacteria use mycotoxins (i.e. peanuts, certain other legumes, corn, soy) to further strengthen the biofilm that houses and protects viruses. The more amyloid-rich food that is consumed, the more pathogens may strengthen their biofilm, placing a strain on the immune system response. This further illustrates one example of the power of the epigenetic expression system 104 in providing useful insight into a pathogenic load, which creates an environment for numerous health problems and requires a unique lens to be understood. In some examples, the epigenetic expression system 104 may output a recommendation that wild game raised in non-inflammatory conditions may lower the risk of harmful health effects as compared to commercially raised meats. Which becomes ever more granular if the individual has genetic tendencies for fat malabsorption, in which case even wild game or seafood that is considered to be rich in fats may be contraindicated, a correlated benefit identified by the epigenetic expression system 104 may include improved digestion, energy, and immunity and enhanced weight loss.

Examples of criteria that may be analyzed and/or weighted by the epigenetic expression system 104 may include the following:

### Gene Implications for Proteins: MTHFR A1298c, CYP Gene Family, HLA Gene Family, ACAT

Fat is the body's main storage of energy in the body. Fat is found in high concentrations in the brain, and thus plays a role in brain structure and cognitive function. Fat metabolism impairment may affect a multitude of bodily organs including the liver, gallbladder, spleen, stomach, brain, endocrine system, and nervous system. When fat metabolism impairment is present, the system of systems 100 also examines the role of stress hormones such as cortisol and epinephrine, mold, and insulin as potential contributors to this issue. In addition, modulating dietary fat consumption becomes imperative for recovery. Impairments in fat metabolism may be linked to insulin resistance, metabolic syndrome, obesity, high cholesterol, atherosclerosis, cardiovascular disease, and stroke.

### Gene Implications of Fat Metabolism: APOE, PEMT, VDR, MTHFR C677T, ACSL1, FADS, ACAT, COMT, OXTR, SUOX, GRHPR, SPP1

Sulfur is a compound found in many foods, pharmaceuticals, and supplements. Examples of foods high in sulfur include cruciferous vegetables, garlic, onion, dried fruits, and eggs. If the CBS enzyme is impaired it may be linked to a build-up of hydrogen sulfide gas (H2S) causing a multitude of gastrointestinal issues. H2S imbalances could be linked to stress, meat consumption, and high sulfur diets, particularly if an individual has genetic predispositions that impair sulfur metabolism. Impaired sulfur processing is also linked with elevated histamine, anxiety and depression, elevated homocysteine, asthma and skin issues.

In addition to conflicting views on animal proteins, and the emphasis on paleo and ketogenic diets, there has been a widespread promotion of sulfur-rich foods such as cruciferous vegetables, egg yolks, and garlic in most health care circles. However, some individuals may have genetic impairments in sulfur processing that require limiting sulfur-containing foods and the introduction of supplements for proper healing. High sulfur diets may degrade the mucosal lining of the stomach leading to inflammation. Sulfur metabolism impairment may also be linked to polymorphisms in a variety of genes such as CBS, SUOX, QDPR, SULT, CYP, and BH4. This genetic predisposition coupled with the pervasive use of herbicides containing glyphosate further impairs sulfur transport and utilization. Glyphosate is an active ingredient commonly used in herbicides and is often sprayed on wheat crops in many countries. Glyphosate may be linked to gut dysbiosis, impaired mineral metabolism, and the inhibition of CYP450 enzymes. Other possible symptoms of sulfur metabolism impairment may include allergies to sulfa-containing medications (bactrim, glutathione, MSM, etc.), reactions to garlic, reactions to eggs, asthmatic symptoms after ingesting preservative-containing foods, strong urine odor after asparagus consumption, and joint pain.

### Gene Implications of Sulfur: CBS, CYP Gene Family, SUOX, SULT, BH4, QDPR

Oxalates, also known as oxalic acids, can be found in high concentrations in certain vegetables, fruits, nuts, grains, and other food sources such as curry (for example, the active ingredient of turmeric0. Diets high in oxalates may be associated with acute and chronic kidney disease, kidney stones, pancreatic inflammation, cardiovascular complications, diabetes mellitus, mental health, and immune system impairment. Oxalates may deposit in the kidneys, lungs, joints, brain, bones, GI tract, thyroid, and blood vessels thus exacerbating inflammation in the given organ. Oxalates may also inhibit calcium and fat absorption which may be linked to calcifications, neurological issues, muscle spasms, and skeletal and bone issues. Bacterial imbalances in the gut microbiota and, once again, glyphosate may put a strain on the body's ability to break down oxalates. Individuals experiencing symptoms of gut dysbiosis may also have underlying oxalate metabolism issues.

### Gene Implications of Oxalates: SUOX, GRHPR, HOGA1, SPP1

In some examples, the Big Four disruptors have the potential to influence one another. In other words, issues with fat metabolism could impact sulfur impairment, and oxalate load and visa versa. Issues with factors outside of the Big Four disruptors such as neurotransmitters, hormones, methylation, liver detoxification, and heavy metals may also bring about issues in more than one of the four big disruptor categories within specific individuals.

A reduction in the consumption of conventional animal proteins, foods that contain mycotoxins, and foods that are rich in sulfur and oxalates, may be identified by the epigenetic expression system 104 for reduction if these substances are affecting genetic expression. Through a detailed inventory emphasizing protein, fat, sulfur and oxalate malabsorption, individuals are categorized into archetypes based on their current state of health and genetic blueprint. Based on these results, recommendations for individualized eating plans and lifestyle practices may be provided by the system of systems 100.

In the illustrated embodiment, the epigenetic expression system 104 further includes the emotional portal 206 which may correspond to data relating to, for example, in-utero trauma, inherited DNA, transgenerational trauma, patterning and programming based on triggers of life experiences, and/or the like. The physical portal 208 may correspond to data relating to, for example, impact to the body through accident, labor, trauma, sports, and/or the like.

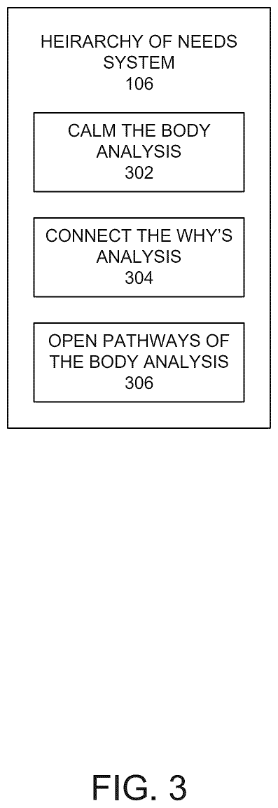

FIG. 3 is a block diagram of the hierarchy of needs system 106 of the system of systems 100 of FIG. 1, according to one or more examples of the disclosure. In some embodiments, the hierarchy of needs system 106 correlates, calculates, and applies weighting factors to provide a recommendation directed to the rebalancing of any health condition.

A common and consistent error that health practitioners make is to suggest a medical, dietary, or supplementation plan that addresses the components of a health condition out of sequence or out of alignment with their genetic blueprint and their current state of health, resulting in a more imbalanced state including, the exacerbation of a disease state, or a “band-aid” effect, wherein the root cause of the condition is masked, but not resolved. This is commonly known as treating the symptom without treating the cause.

To generate a successful and bio-individual plan 110 to improve the health of an individual, the heirarchy of needs system 106 is powerful in establishing correlations and solutions. In some embodiments, bio-individual plan 110 is advised by the hierarchy of needs system 106 as to the order in which components of bio-individual plan 110 are structured. In some embodiments, the hierarchy of needs system 106 provides an order for treatment to reduce the chance of relapse, less-effective treatment, conflict of treatment, etc.

For example, if an individual experiencing hormonal imbalance begins supplementing with bio-identical hormones before addressing vital structures like the HPA axis, the liver detoxification pathways, fat metabolism efficiency, pathogenic activity like candidiasis, or gut integrity where elevated beta-glucuronidase may be recycling hormones thereby increasing the estrogen burden, they risk intensifying their symptoms and worsening their underlying condition. A sequence of treatment, built by the hierarchy of needs system 106 may add upon itself and take into account the sequence of rebalancing stages that the body may need to undergo to produce more effective and sustainable results based on a priority arrangement.

In some embodiments, the hierarchy of needs system 106 may include a calm the body analysis 302. When the autonomic nervous system is in a state of hyper-vigilance or constant activation, it can be challenging or impossible for any body structure to perform optimally. To address imbalance in the body, the body may first be restored to a state of calm. Identifying actions to soothe the HPA axis can create a cascade of positive results, including improving blood sugar regulation, hormone regulation, neurotransmitter regulation, supporting gut integrity, nutrient absorption and emotional health, to name a few. If the system of systems 100 is directed to balance a pathogenic load in the state of overactive autonomic nervous system response, it will be much more rigorous to balance that pathogenic load until this system is calmed as an overactive HPA axis can increase pathogenic strength.

The hierarchy of needs system 106 may also include a connect the why's analysis 304: The body may enter a state of imbalance or disease in response to a specific trigger, or triggers. This could be anything from the introduction of a new medication, an environmental change such as moving to a new home with a mold problem, an improper diet, exposure to a pathogen, or an emotional trauma such as a family death or divorce. It is vital to listen closely to a client's story and to ask the right questions. Providing context for the disease state often illuminates its root cause. The “Connect the Why's” analysis intakes the client's “story” to create a corelative interconnection to assist in generating the bio-individual plan 110.

The hierarchy of needs system 106 may also include an open pathways of the body analysis 306. The human body has detoxification channels that allow us to pass, or get rid of, toxins, bacteria and pathogens. When these channels become blocked or imbalanced, it can lead to toxic accumulation and create a tipping point for the body. However, if a plan is created which starts pulling toxins or attacking a pathogen before an individual's detoxification pathways are open, they are likely to undergo enterohepatic recirculation. Biliary acids from the liver break down toxins which pass through the small intestine, where they are reabsorbed and transported back to the liver in what may be termed a “detox retox cycle.” This result can intensify symptoms and lead to the failure of a health protocol. By analyzing and detecting the pathway status of the body, in the open pathways of the body analysis 306, a plan can be generated for opening affected pathways. Opening these detoxification and metabolic pathways—in some embodiments, with a focus on the skin, liver, kidneys, lymph and gut—the system of systems 100 can generate a successful bio-individual plan 110 and begin to effectively clear the body of toxins and disease.

The hierarchy of needs system 106 can be the difference between a successful health protocol and a worsening disease state. In some embodiments, to facilitate healing, the nervous system may be calmed, the root causes identified, and the detoxification pathways restored.

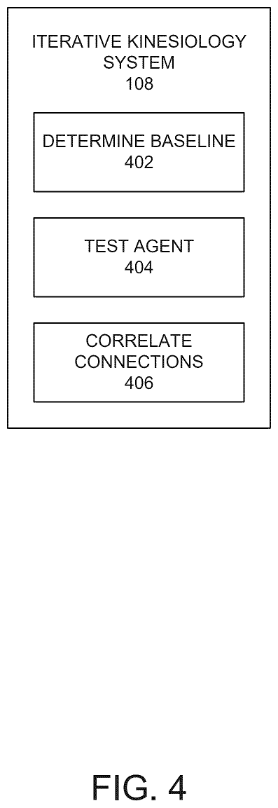

FIG. 4 is a block diagram of the iterative kinesiology system 108 of the system of systems 100 of FIG. 1, according to one or more examples of the disclosure. The system of systems 100 applies the iterative kinesiology system 108 to identify physiological imbalances and generate a food and supplement component of the bio-individual plan 110.

Humans have a central nervous system that runs through the tissues, as well as fluids like blood and lymphatic fluids that carry not just chemical and electrical information, but also electromagnetic information. Within the body, cells communicate in many different ways; not just chemically and electrically (which are well-documented), but also electromagnetically.

Not unlike an electrocardiogram (EKG), applied kinesiology measures closed-circuit electrical signals as the body responds to different stimuli. The iterative kinesiology system 108 builds on conventional applied kinesiology. The system of systems 100 implements iterative kinesiology to drive the question answered by the body to drive the next question of the body measured by the system of systems 100. The iterative kinesiology system 108 may apply pressure to the arm to determine a baseline 402. Determining the baseline 402 may include measuring and recording a strength test. The iterative kinesiology system 108 may include a second measurement and recording with a test agent 404. The test agent 404 may include a glass vial containing a stimulating agent. The stimulating agent may include a material having an electromagnetic signature of a food or pathway (such as methylation) or a pathogen (such as E. coli). The test agent 404 may be placed within a field of the person being tested. In some embodiments, if a laxity or weakness is detected relative to the baseline 402, it may be determined as a signal from the body that there is an imbalance within the body in relationship to the test agent 404. In some embodiments, the test agent 404 may also be correlated with a positioning of the test agent 404 relative to the body of the person being tested.

In some embodiments, the system of systems 100 employs the iterative kinesiology system 108 to identify connections not only between test agent 404 and the body as a whole, but between test agent 404 and specific structures and systems of the body. The iterative kinesiology system 108 may include correlating connections 406 which may be based on the measured differences between the baseline 402 and the test agent 404 to provide insight into a wide range of health conditions with hyper-granularity and real-time feedback from an individual's body, capturing the signaling impairments that drive symptomatology and illuminating systemic imbalances, creating an informational dataset that builds upon the results of each consecutive test, establishing a line of communication that works from the inside out.

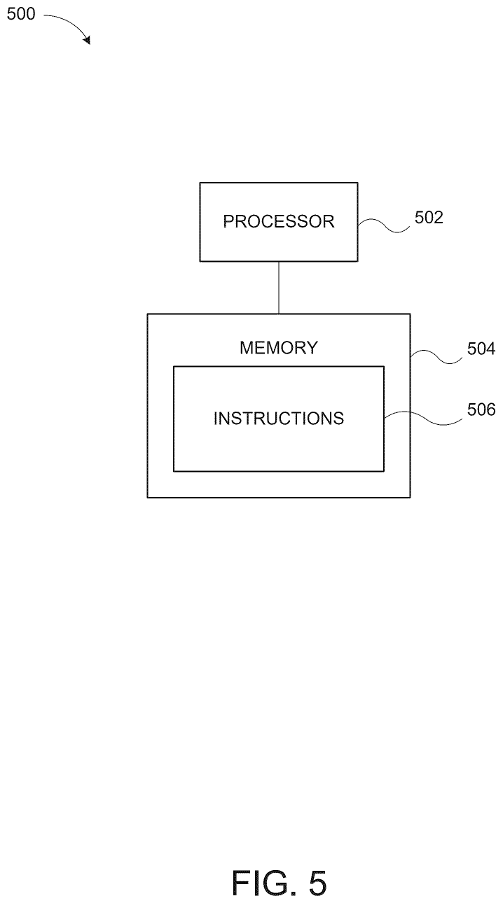

FIG. 5 is a block diagram of an apparatus 500, according to one or more examples of the disclosure. The apparatus 500 may be a processor-built resource built around one or more processors 502 and a memory 504. The one or more processors 502 may be used for controlling the general operations of the apparatus 500, as well as handling and/or processing of the data generated and/or received by at least one of the iterative intake 102, the epigenetic expression system 104, the hierarchy of needs system 106, and/or the iterative kinesiology system 108. The one or more processors 502 may be any suitable processor-based resource. They may be, but are not limited to, a central processing unit (“CPU”), a hardware microprocessor, a multi-core processor, a single core processor, a field programmable gate array (“FPGA”), a controller, a microcontroller, an application specific integrated circuit (“ASIC”), a digital signal processor (“DSP”), or other similar processing device capable of executing any type of instructions, algorithms, or software for controlling the operation and performing the functions of the one or more processors 502. In some embodiments, the one or more processors 502 may comprise a processor chipset including, for example and without limitation, one or more co-processors.

The memory 504 may be a single memory device or one or more memory devices at one or more memory locations that may include, without limitation, one or more of a random-access memory (“RAM”), a memory buffer, a hard drive, a database, an erasable programmable read only memory (“EPROM”), an electrically erasable programmable read only memory (“EEPROM”), a read only memory (“ROM”), a flash memory, hard disk, various layers of memory hierarchy, or any other non-transitory computer readable medium. The memory 504 may be on-chip or off-chip depending on the implementation of the one or more processors 502. The memory 504 may be used to store any type of instructions 506 and data associated with algorithms, processes, or operations of or for the iterative intake 102, the epigenetic expression system 104, the hierarchy of needs system 106, and/or the iterative kinesiology system 108, or combinations thereof.

Data may be acquired and processed by the processor 502, or data may be transmitted from the processor 502 and or the memory 504 to a remote location for use and/or analysis, or some combination thereof. As used herein and in this context, “remote” means outside the physical presence of the processor 502. Conversely, “local” means in the physical presence of the processor 502. Such remote locations may include, without limitation, central computation or storage in another location or facility, or in a computing or storage cloud located, at least in part, at another facility. Those skilled in the art having the benefit of this disclosure may appreciate still other variations on this theme.

In some embodiments, the apparatus 500 may implement an artificial intelligence (AI) or other machine learning model to generate the bio-individual plan 110. For example, the machine learning model may be stored on the memory 504 and executed by the processor 502. The machine learning model may be trained using a training data set. The training data set may include a collection of medical treatments and/or outcomes, patient historical data, health correlations, outcome databases, patient data, and the like. The model may be trained to identify patterns from the training data set. Once trained, the model may be able to generate the bio-individual plan 110 specific to a particular patient based on patterns identified in data generated and/or output by at least one of the iterative intake 102, the epigenetic expression system 104, the hierarchy of needs system 106, and/or the iterative kinesiology system 108. The machine learning model may apply a weighting to the data received from at least one of the iterative intake 102, the epigenetic expression system 104, the hierarchy of needs system 106, and/or the iterative kinesiology system 108.

In some embodiments, the machine learning model may be a self-learning model using artificial intelligence (AI) to form determinations based on the data received from at least one of the iterative intake 102, the epigenetic expression system 104, the hierarchy of needs system 106, and/or the iterative kinesiology system 108. The machine learning model may, for example, include medical analysis algorithms. The machine learning model may rely, at least partly, on inference, conduction, and other pattern recognition, once trained, to generate the bio-individual plan 110. The machine learning model may, for example, determine a pattern based on patient intake information, medical history, laboratory, radiology, other medical information, patient instruction/directive, or the like.

In some embodiments, the apparatus 500 may implement a diagnosis protocol, a therapy protocol, or other standards or guidelines-based protocol. Such protocols may be based upon standardized medical guidelines and algorithms, physician rules or recommendations, and/or known parameters from a medical expert in a relevant field.

The apparatus 500 may be in communication with a network or other communication structure to provide the bio-individual plan 110 to one or more recipients. For example, the apparatus 500 may transmit the bio-individual plan 110 to the patient, a caregiver (e.g., nurse, diagnostician, surgeon, emergency personnel, etc.).

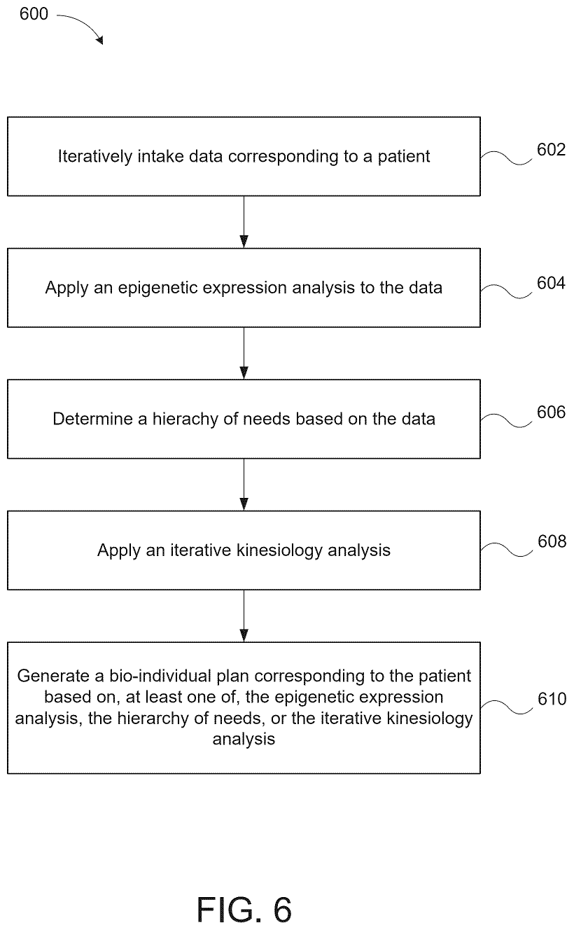

FIG. 6 is a flowchart depicting a method 600 for implementing the system of systems 100 of FIG. 1, according to one or more examples of the disclosure. In some embodiments, the method 600 includes, at block 602, iteratively intaking data corresponding to a patient. In some embodiments, the iterative intake process may include a questionnaire, exam, history intake, or the like to intake data corresponding to the current health, health history, and the like for a patient. The iterative intake may be static or dynamic. For example, a set of questions may be generated prior to the intake iterative intake and administered without adaptation. A dynamic approach may be applied which generates and adapts the questions and intake based on data received through the iterative intake.

The method 600 may include, at block 604, applying an epigenetic expression analysis to the data. For example, the epigenetic expression system 104 may identify and correlate genetically expressed imbalances and impairments, identifying their root cause, and isolating effective solutions for rebalancing an individual at the epigenetic blueprint level.

The method 600 may include, at block 606, determining a hierarchy of needs based on the data. For example, the hierarchy of needs system 106 may correlate, calculate, and apply one or more weighting factors to provide an output relevant to the bio-individual plan 110.

The method 600 may include, at block 608, applying an iterative kinesiology analysis. For example, the iterative kinesiology system 108 may identify physiological imbalances and generate a food and supplement component of the bio-individual plan 110.

The method 600 may include, at block 610, generating a bio-individual plan corresponding to the patient based on, at least one of, the epigenetic expression analysis, the hierarchy of needs, or the iterative kinesiology analysis. For example, the system of systems 100 may implement an iterative intake 102 to feed data to three interrelated processing components: an epigenetic expression system 104, a hierarchy of needs system 106, and an iterative kinesiology system 108. The output of the system of systems 100 is a bio-individual plan 110.

In some embodiments, the method 600 may be implemented in software or a combination of software and hardware. Some examples in the present disclosure may also be directed to a non-transitory computer-readable medium storing computer-executable instructions and executable by one or more processors of the computer via which the computer-readable medium is accessed. A computer-readable media may be any available media that may be accessed by a computer. By way of example, such computer-readable media may comprise RAM, ROM, EEPROM, CD-ROM or other optical disk storage, magnetic disk storage or other magnetic storage devices, or any other medium that may be used to carry or store desired program code in the form of instructions or data structures and that may be accessed by a computer. Disk and disc, as used herein, includes compact disc (CD), laser disc, optical disc, digital versatile disc (DVD), floppy disk and Blu-ray® disc where disks usually reproduce data magnetically, while discs reproduce data optically with lasers.

Note also that the software implemented aspects of the subject matter claimed below are usually encoded on some form of program storage medium or implemented over some type of transmission medium. The program storage medium is a non-transitory medium and may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The claimed subject matter is not limited by these aspects of any given implementation.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Obviously, many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims

What is claimed is:

1. A method comprising:
iteratively intaking data corresponding to a patient;
applying an epigenetic expression analysis to the data;
determining a hierarchy of needs based on the data;
applying an iterative kinesiology analysis; and
generating a bio-individual plan corresponding to the patient based on at least one of the epigenetic expression analysis, the hierarchy of needs, or the iterative kinesiology analysis.

2. The method of claim 1, wherein the epigenetic expression analysis comprises identifying a cause of a symptom described in the data corresponding to the patient.

3. The method of claim 2, wherein identifying the cause comprises identifying phenotypes or inheritable shifts in gene expression apart from those that the structure of the core genotype or DNA.

4. The method of claim 1, wherein applying the hierarchy of needs comprises applying a weighting component to the data corresponding to the patient.

5. The method of claim 1, wherein the iterative kinesiology analysis comprises measurement of a response to a stimulus.

6. The method of claim 1, wherein the iterative kinesiology analysis comprises creation of a map of symptomatic expression and signaling impairment of the patient.

7. A system of systems, comprising:
an iterative intake to collect data corresponding to a patient;
an epigenetic expression system to analyze the data;
a hierarchy of need systems to calculate a hierarchy based on the data;
an iterative kinesiology system to applying an iterative kinesiology analysis; and
a bio-individual plan corresponding to the patient based on at least one of the epigenetic expression analysis, the hierarchy of needs, or the iterative kinesiology analysis.

8. The system of claim 7, wherein the epigenetic expression system is configured to identify a cause of a symptom described in the data corresponding to the patient.

9. The system of claim 8, wherein the epigenetic expression system is configured to identify phenotypes or inheritable shifts in gene expression apart from those that the structure of the core genotype or DNA.

10. The system of claim 7, wherein the hierarchy of needs system is configured to apply a weighting component to the data corresponding to the patient.

11. The system of claim 7, wherein the iterative kinesiology system is configured to measure a response to a stimulus.

12. The system of claim 7, wherein the iterative kinesiology system is configured to create a map of symptomatic expression and signaling impairment of the patient.

13. An apparatus, comprising:
a processor;
memory in communication with the processor; and
instructions stored in the memory, the instructions executable by the processor to:
iteratively intake data corresponding to a patient;
apply an epigenetic expression analysis to the data;
determine a hierarchy of needs based on the data;
apply an iterative kinesiology analysis; and
generate a bio-individual plan corresponding to the patient based on at least one of the epigenetic expression analysis, the hierarchy of needs, or the iterative kinesiology analysis.

14. The apparatus of claim 13, wherein applying the epigenetic expression analysis comprises identifying a cause of a symptom described in the data corresponding to the patient.

15. The apparatus of claim 14, wherein identifying the cause comprises identifying phenotypes or inheritable shifts in gene expression apart from those that the structure of the core genotype or DNA.

16. The apparatus of claim 13, wherein applying the hierarchy of needs comprises applying a weighting component to the data corresponding to the patient.

17. The apparatus of claim 13, wherein the applying iterative kinesiology analysis comprises measuring a response to a stimulus.

18. The apparatus of claim 13, wherein applying the iterative kinesiology analysis comprises creating a map of symptomatic expression and signaling impairment of the patient.

19. The apparatus of claim 13, wherein the bio-individual plan is based on a correlative linking between at least one of the epigenetic expression analysis, the iterative kinesiology, and the intake data.

20. The apparatus of claim 13, wherein applying the epigenetic expression analysis comprises applying at least one of a pathogenic analysis, an environmental analysis, an emotional analysis, and a physical analysis.
